(19) United States
(12) Patent Application Publication
(10) Pub. No.: US 2023/0191072 A1
(43) Pub. Date: Jun. 22, 2023

(54) BREATHING SYSTEM DEVICE FOR CO2 REMOVAL BASED ON AN ELECTRO-CHARGING AND DISCHARGING METHOD

(71) Applicant: Kristof Braem, Nieuwerkerken-Aalst (BE)

(72) Inventor: Kristof Braem, Nieuwerkerken-Aalst (BE)

(21) Appl. No.: 17/924,790

(22) PCT Filed: May 11, 2021
(86) PCT No.: PCT/EP2021/062530

§ 371 Date: Nov. 11, 2022

(30) Foreign Application Priority Data
May 11, 2020 (BE) BE2020/5319

(51) Int. Cl. A61M 16/22; B01D 53/32; B01D 53/04; A61M 16/01

(52) U.S. Cl. CPC A61M 16/22; B01D 53/326; B01D 53/04; A61M 16/01; B01D 2259/818; B01D 2253/202; B01D 2256/12; B01D 2257/504; B01D 2259/4533

(57) ABSTRACT

The invention relates to a reusable device for CO2 removal, suitable for use in a breathing system, as part of an anaesthesia arrangement, and based on an electro-charging and discharging method. The invention is related to holders, containers and so-called canisters including any subparts of such devices and methods of operating used in an anaesthesia arrangement, wherein the CO2 removal takes place.

### TECHNICAL FIELD

The invention generally relates to the field of anaesthesia, and in particular to purification of expiration gases during anaesthesia. More in particular, the invention relates to a regenerative device for CO2 removal, and to methods of regeneration of such a device for multiple use thereof. CO2 is removed from a (potential patient) gas stream, suitable for use in a breathing system, and for example being part of an anaesthesia arrangement. The invention is related to holders, containers and so-called canisters including any subparts of such devices and methods of operating used in an anaesthesia arrangement, wherein the CO2 removal takes place.

### BACKGROUND OF THE INVENTION

Anaesthesia ventilation is a closed or semi-open to closed system. This means that part of the ventilation exhaled by the patient will be reused for inspiration and ventilation of the patient. When reusing these gasses, the CO2 exhaled by the patient, has to be removed from the system.

Currently and mainly applied CO2 removing technique is based on the use of soda lime as an absorbent for CO2. Although recently, there is a tendency to move away from this technique because of the drawbacks of chemical absorbers, including for example the production of compounds being harmful to patients leading to increased cost and environmental impact, as well as the daily disposal of compound special waste. Moreover, the disposal of chemical granulates is becoming a growing environmental concern. As a sustainable alternatively, a membrane technique to filter CO2 from anaesthetic circuits can be used. This rather new technique relying on a polymeric membrane core for selectively allowing CO2 to leave the breathing system, while maintaining anaesthetic vapor in the circuit, is for the moment still more in experimental phase.

The use of soda lime, as an absorbent of the CO2, makes rebreathing possible, thus conserving gases and volatile agents, decreasing of pollution, and avoiding hazards of carbon dioxide rebreathing. Soda lime activator is NaOH or KOH. Silica and kieselguhr are added as hardeners. Indicators for soda lime (such as ethyl violet) are colourless when fresh, and purple when exhausted, because of pH changes in the granules. As a result, soda lime granules will change colour (to purple) when exhausted, being an indication (when purple) that they need to be replaced. Typically, every 2 days, one has to replace the soda lime with new granules.

The use of soda lime has many disadvantages, such as for example their reaction with other gasses. E.g. sevoflurane is unstable in soda lime, producing Compound A (lethal at 130-340 ppm, or renal injury at 25-50 ppm in rats; but incidence of toxic [hepatic or renal] or lethal effects in millions of humans are comparable to desflurane). Compound A concentrations of 25-50 ppm are easily achievable in normal clinical practice. Sevoflurane package insert recommends avoiding FGF at 1 L/min for no more than 2 MAC-Hours (2 L/min FGF can be used indefinitely). Further, carbon monoxide is produced by (desflurane>enflurane>isoflurane)>>(halothane=sevoflurane), whereas the situation being worse in dry absorbent. It is thus important to turn oxygen off at end of a patient case, change the absorbent regularly, e.g. change if FGF left on over the weekend or overnight, and use low flows, which will tend to keep granules moist.

Special and more expensive soda lime is already available on the market, to avoid the compound A production. However, still small part of the anaesthetic agent will react, resulting in a higher consumption than usually needed. The strong bases (NaOH, KOH which function as activators) have been convincingly implicated in the carbon monoxide problem with the ethyl-methyl ethers, and the generation of Compound A by sevoflurane, leading to very often replacement. Hence, waste management and the degree pollution have been considered. As a conclusion, current available solutions are rather expensive when calculated total cost per year/per unit.

In addition, other problems that can occur with soda lime are for example, the challenge in sizing a compromise between absorptive capacity and resistance to airflow, and the resistance of a full canister being <1 cm H2O at 60 L/min flow through the canister. Further, inhaled dust is caustic and irritant. Moreover, one may see exhaustion without colour change, due to channelling or inactivation of indicator along the canister walls by UV light.

In general, in present devices for CO2 removal in anaesthetic arrangements as described above, soda lime granules are used, being removed and disposed of after their use. From an environmental point of view, such approach is no longer acceptable.

However, considering alternative more environmental friendly techniques for CO2 removal for use in the anaesthetic field is hampered by the observation that this field has specific requirements in terms of bio-compatibility for use with humans, that reusability requires autoclavability and one should consider particular use requirements in terms of typical (up to low) concentrations of CO2 in the gas stream and pressure restrictions when typical flow rate for this field application is used. Removal of gasses of a particular kind in an anaesthesia breathing context are known like in US2010258117 related to recycling of Xenon.

While US2008202341 emphasizes the importance of carbon dioxide removal in general and anaesthesia in particular, its use of an ionic membrane and/or by liquid impregnated cathode and anode makes it inappropriate for the particular use of anaesthesia. Carbon dioxide removal is also referred to in US2010180889, going along with oxygen generation for use in battlefield applications where oxygen requirements may be extreme.

### AIM OF THE INVENTION

It is the aim of the invention to provide an environmentally friendly technique for CO2 removal for and adapted to the particular anaesthetic field.

### SUMMARY OF THE INVENTION

In a first aspect of the invention a device for CO2 removal from a gas stream, in particular an expiration or breathing gas stream (in the presence of an anaesthetic agent), suitable for use in a breathing system, part of an anaesthesia arrangement, is provided. The device comprises a holder, adapted for fitting in such breathing system, and a container for CO2 removal from a gas stream, wherein the container is adapted for fitting into the holder. According to an embodiment, the device consists of a holder, adapted for fitting in such breathing system, and a container for CO2 removal from a gas stream, wherein the container is adapted for fitting into the holder. The CO2 removal of the container is based on an electro-charging and discharging method. Whenever the device is placed in the breathing system, and the charging mode of the device is switched on, CO2 will be removed from the gas stream and will be captured and absorbed by the container. After a while, the container gets saturated with CO2 and has to be switched off for charging and switched on to discharging mode. This way the CO2 can be repelled from the container until it is free of CO2 again, and ready for charging reuse in the breathing system. The device is again operational for removing CO2 from the gas stream when switched on in charging mode. This iterative process can be repeated again.

According to an embodiment, the container comprises one or more plates, each comprising of at least two electrodes arranged for charging and/or discharging the one or more plates. The one or more plates can be adapted for absorbing CO2 when the electrodes being charged, and for releasing CO2 when the electrodes being discharged. The electrodes may comprise of an active electrode, and a counter electrode, being all or not separated by a dielectric or isolating separator. The device may comprise means for providing electricity to the active and/or counter electrode. Further, the device may comprise electronic means such as an electronic circuit to thereby control the charging and/or discharging operation of the electrodes. According to an embodiment, the device including holder, container, plates and electrodes are autoclavable and biocompatible for use with humans.

According to an embodiment, the device comprises at least two plates and may be adapted for having a pressure less than 2 mbar at a nominal flow rate between 20 to 65 L/min, wherein the distance d between the plates the electrodes thereof is selected therefore.

According to an embodiment, the surface of the electrodes, i.e. the active electrode in particular, is selected to react with CO2 in the gas stream. The surface of the electrodes may be covered with a polymer, arranged in a design for optimal surface use like a honeycomb structure, wherein the polymer is containing material for attracting CO2 such as for example (anthra)quinone. The surface of the electrodes may be covered with a polymer, arranged in a design for optimal surface use like a honeycomb structure, wherein the polymer is comprising material for enhancing the conductivity such as for example carbon nanotubes.

According to an embodiment, the device is adapted for being capable to being operable for concentrations as low as around 400 ppm of CO2, by selecting of the amount of (anthra)quinone and the total surface (for CO2 removal) applicable in the container. This can be achieved depending on the of amount of (anthra)quinone and the total surface (for CO2 removal) applicable in the canister. The device can also be adapted for being operable for at least one day or 24 hours, preferably one (working) week of 5-7 days, wherein the operability in time can be chosen, designed or selected by means of the amount of plates used in the container for CO2 removal. Furthermore, the device can be adapted for being operable for at least 2000 charging-discharging cycles, preferably 5000 charging-discharging cycles with less than 30% efficiency loss of the charging-discharging operation.

The device for CO2 removal is designed via its selection of materials for CO2 absorbance, the amount of those materials used and the arrangement thereof, to be capable to handle typical (low) concentrations and/or to have a suitable cycle of use (for this application) while (given the environmental goal) also a certain durability. For instance, the amount of plates, and the amount of a (specific) polymer (provided with additional elements) may play a role in the design of the CO2 removal device, more in particular regarding the CO2 absorption capacity thereof. The design of the plate or electrode surface onto which a polymer is applied, may further be of importance. Moreover, in relation to the use requirements, the arrangement of the materials (for instance in terms of distance between plates) takes into account conditions on experienced pressure (by the patient).

In a second aspect of the invention a two-mode method for operating the device for CO2 removal in accordance with first aspect, is provided. The two-mode method comprises an electro-charging and discharging method for operating the device for CO2 removal. The comprises of the following steps. Firstly, when the device is installed in the gas flow circulation of a breathing system, part of an anaesthesia arrangement, from which it should remove the CO2, electricity of a first polarity is provided to the container in order to enable charging operation, meaning CO2 is captured and absorbed by the container under electricity. Secondly, when the device is removed from the gas flow circulation, electricity of a second polarity (different or reverse of the first policy) is provided to the container such that the device can be discharged, meaning CO2 is repelled or removed again from the container under electricity.

### BRIEF DESCRIPTION OF THE DRAWINGS

FIG. 1 illustrates schematically an embodiment of a device comprising an electrochemical plate and corresponding electrodes for CO2 removal from a gas stream, suitable for use in a breathing system, part of an anaesthesia arrangement, in accordance with the invention.

FIG. 2 shows a further embodiment of a device comprising two electrochemical plates with respective electrodes for CO2 removal from a gas stream, suitable for use in a breathing system, part of an anaesthesia arrangement, in accordance with the invention.

FIG. 3 illustrates a more detailed version of the embodiment of FIG. 1 including means to provide electricity to the CO2 removal device, in accordance with the invention.

FIG. 4 illustrates an exemplary embodiment of (a) charging, and (b) discharging process of a CO2 removal device with a plate defined by electrodes and separators there in between, and wherein means for providing electricity to the electrodes are foreseen, in accordance with the invention.

FIG. 5 illustrates an embodiment of the use of the CO2 removal device in a breathing system context, in accordance with the invention.

### DETAILED DESCRIPTION OF THE INVENTION

With the present invention, a traditional soda lime canister is replaced by a canister, holder or device containing a stack of electrochemical plates absorbing carbon dioxide from the circuit breathing gasses (or other gas stream) passing over its electrodes or e.g. a stack of electrodes, as the electrodes of the plates are charged up, and then releasing the CO2 gas as the electrodes of the plates are discharged. Hence, a new way of removing CO2 in the breathing system (wherein a stream of rebreathing gasses of the patient and/or fresh gas mixture is circulated) of an anaesthesia arrangement or workstation, is provided by means of an electro-charging and discharging system. According to an embodiment, CO2 is removed from a gas mixture of air, oxygen, nitrous oxide and/or anaesthetic agent (isoflurane, enflurane, desflurane, halothane and sevoflurane). This mixture may be either as combination of these different gasses, as well as in combination with individual gasses.

While charging, an electrochemical reaction takes place at the surface of the electrodes (or each of a stack of electrodes) and will attract the CO2 in the breathing circuit, meaning that the electrodes (or the surfaces thereof) will capture and absorb the CO2 present in the gas stream passing by. The electrodes may have a natural affinity for carbon dioxide and may readily react with its molecules in the gas stream, even when it is present a very low CO2 concentrations (down to the roughly 400 parts per million currently found in the atmosphere). The whole system operates at room temperature and normal pressure. The electrodes can be coated with a polymer, containing (anthra)quinone and composited with carbon nanotubes. The reverse reaction will take place while discharging.

According to an embodiment, the electrochemical plates comprise of one or more active coated electrodes, one or more counter electrodes and a separator between each active and counter electrode respectively. In an embodiment, the active electrodes and counter electrodes are placed or separated far enough from each other, such that a separator in between them is no longer necessary. In an embodiment, the complete electrochemical cell consists of an active electrode (or stack of electrodes), a counter electrode and/or a separator (depending on mounting position). By means of example, a graphene sheet can be used as counter electrode. The system can work at virtually any CO2 concentration level, even down to the roughly 400 parts per million currently found in the atmosphere.

The canister containing the electrodes can be placed or located in the breathing system on the inspiratory side, as well as the expiratory side.

The number of electrode plates or the total electrode surface (e.g. given by a stack of electrodes) used will determine the capacity of the CO2 absorbing. According to an embodiment, the distance/openings between the different plates (or 1 plate in the form of honeycomb) is between 0.8 mm and 5.4 mm.

With the present invention, a reusable system for CO2 removal can be provided for at least 2000 charging-discharging cycles, with less than 30% efficiency loss of that time. The full assembly of the system, meaning all parts, subparts including the materials they are made of, being not only reusable, but is moreover autoclavable and biocompatible for use with humans. By means of example, materials such as Makralon 2458, and Valox Resin HX420HP are applicable. There is no need of daily or weekly replacement, as the system can be charged and discharged for e.g. 5000 charging-discharging cycles with less than 30% efficiency loss. Having such amount of charging-discharging cycles, the system in accordance with the invention is much more sustainable, and has a significant life time as compared to the art.

The electrodes or stack of electrodes can be mounted in a container or canister, with sufficient distance between the electrodes, such that the breathing gasses can pass without causing more than 1 cm H2O pressure at a flow of 60 litres/min.

The canister or container for CO2 removal can be placed in the circle system (on inspiratory side or expiratory side), as an add on by means of e.g. 22 mm Male connector output, and 22 mm Female connector input. The CO2 removal system in accordance with the invention can also be mounted into the existing absorber canister of the respective manufacturer.

According to an embodiment, an existing canister of anaesthesia arrangement or workstation can be used (or new one made compatible), comprising the CO2 removal system in accordance with the invention, such that this CO2 removal system can be connected at same position of current soda lime canister.

### DETAILED DESCRIPTION OF THE DRAWINGS

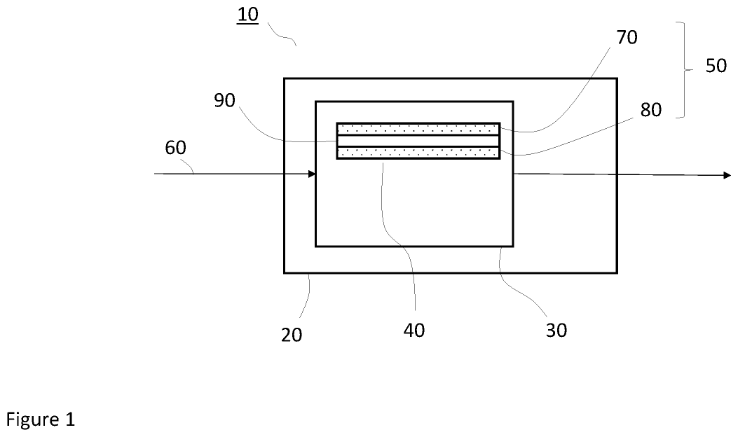

FIG. 1 illustrates schematically a device 10 for CO2 removal from a gas stream 60, suitable for use in a breathing system, part of an anaesthesia arrangement. The device comprises a holder 20, adapted for fitting in the breathing system and a container 30 for CO2 removal from a gas stream 60, adapted for fitting in said holder. The container 30 removes CO2 from the gas stream 60 passing through, based on an electro-charging and discharging method. For enabling this, the container 30 in FIG. 1 comprises a plate 40, e.g. an electrochemical plate, comprising of electrodes 50, amongst which for example an active electrode 70 and a counter electrode 80, being separated from each other by means of a dielectric or isolating separator 90. The charging of the device 10 for absorbing CO2 from the gas steam 60 takes place when charging the active electrode 70 by means of providing electricity there through. According to an embodiment, the surface of the active electrode 70 is provided with material, e.g. a polymer, for attracting CO2. For the electricity provided, one may use for instance voltages of 1-3V. Discharging of the device 10 occurs when the electricity going through the active electrode changes polarity, and thus the CO2 flow is reversed, i.e. instead of CO2 being attracted by the active electrode 70, the CO2 is now being repelled.

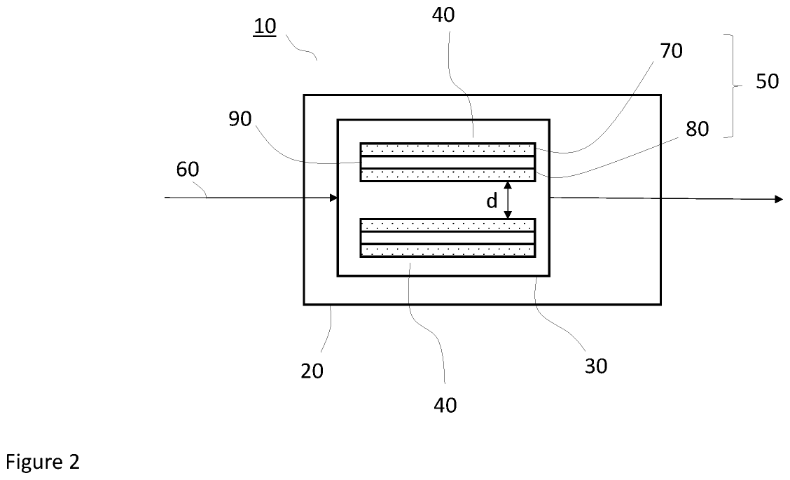

FIGS. 2 shows a further embodiment of the device of FIG. 1, wherein two plates 40 comprising electrodes 50 are drawn, adapted for absorbing CO2 when the device 10 being charged and releasing CO2 when the device 10 is discharged. Between the plates 40, or the electrodes 50 thereof, a distance d is provided. The distance d between the plates 40 plays a role in relation to the pressure requirements for given nominal rates of the gas stream 60 passing between the plates 40, or the electrodes 50 thereof. The distance d between the plates 40 is for example 2 mm, with a minimum of 14 mm total distance available within the container 30 or canister. According to an embodiment (not shown), by means of example 6 to 8 plates 40 could be provided at 2 mm distance from each other within a single container 30. Pressure resistance on the inspiratory side of the canister, may be up to 0.9 cm H2O at peak flow between 20 to 65 litres a minute. With active canister in the breathing system, resistance test of the complete system, including the inventive canister, complies to MDR standards (total maximum resistance lower than 6 cm H2O). The canister can be put on the inspiratory side of the circle anaesthesia rebreathing system, as well as on the expiratory side. Total content of the canister may be for example 1.8 litre (but can be changed to other sizes as well). Pressure in the circuit (and the absorber device) may be between 0 to 120 cm H2O, without any influence on the functionality of the CO2 absorption. The complete canister set is autoclavable, e.g. 134° c. steam sterilization, 8 minutes cycle.

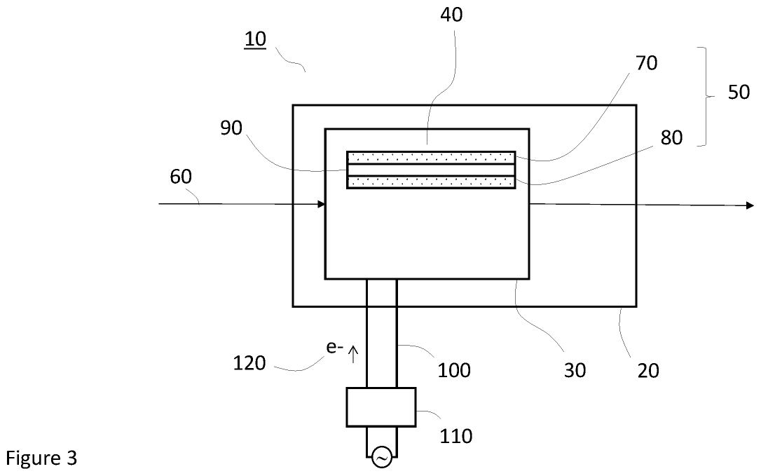

FIG. 3 shows schematically the electricity 120 being provided to the device 10 of FIG. 1, using means 100 being for example an electrically conducting cable or wire. The means 100 is on one hand connected to the container 30 within the holder 20 of the device 10. More in particular (not shown), the means 100 is connected to the electrodes 50, e.g. the active electrode 70 of the plate 40 for charging the electrodes 50 thereof. On the other hand, the means 100 is connected to a power supply, and an electronic means 110 such as an electronic circuit including e.g. an on-off switch for controlling the electro-charging operation of the device 10.

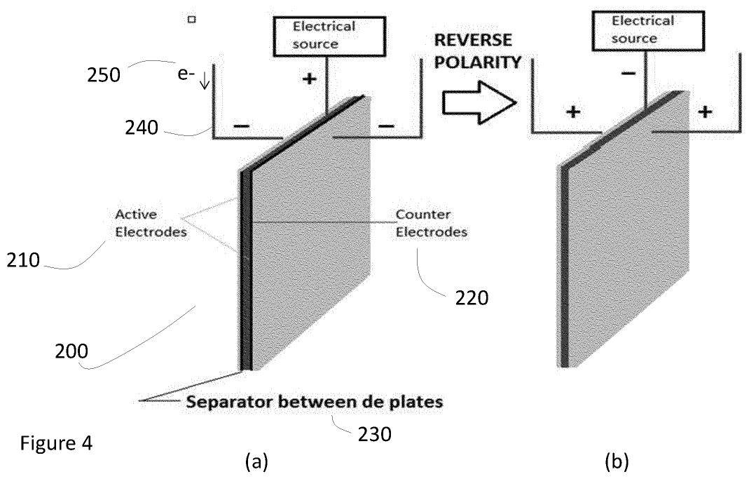

FIG. 4 illustrates an exemplary embodiment of part of the device for CO2 removal in accordance with the invention. A plate 200 is defined by electrodes, in particular an active electrode 210, on the outer side of the plate 200, and a counter electrode 220, on the inner side of the plate 200. The electrodes 210, 220 are separated from each other by a separator 230 having an electrical isolating function. Means 240 e.g. electrical cables or wires, for providing electricity 250 to the active electrodes 210 and/or the counter electrode 220 are foreseen. FIG. 4 also illustrates the two modes of operating for the embodiment wherein in a first mode, depicted in FIG. 4 (a) one provides electricity of a first polarity to charge, and thus removing CO2 from the gas stream wherein the device is immersed, while the CO2 is captured by the active electrodes 210 of the plate 200 and for example being absorbed onto their outer surfaces. The first polarity is indicated by means of the arrow next to e−. A second mode is depicted in FIG. 4 (b). The device is now removed from the gas stream, as for instance being saturated by CO2, and hence ready to be discharged out of the breathing system. Here electricity 250 is provided of a second polarity being different or reverse of the first policy, as indicated by means of reverse arrow next to e−. With this second polarity of electricity 250, the device is now discharged and hence CO2 stored in the device (as being captured thereby) is being released.

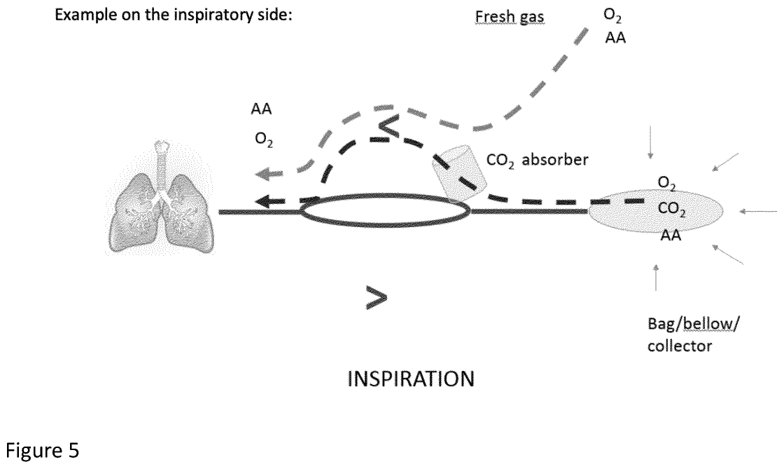

FIG. 5 illustrates the use of the invention in a breathing system context. It is worth emphasizing that the holder or container, also called canister in the field, is or can be provided with suitable connectors of different types (male, female) with typical dimensions of 22 mm diameter. According to this embodiment, the CO2 removal device is placed at the inspiratory side, although it could also be provided at the expiratory side.

According to an embodiment (not shown) the breathing system comprises of at least two CO2 removal devices in accordance with the invention. While one of those devices is switched on for charging, the other one could simultaneously be switched off for charging, and e.g. switched on for discharging. This way, the containers or canisters can alternatingly charge and discharge in parallel, and in a synchronous manner, meaning while one is charging, the other one is discharging at the same time. This time could be for instance one day (or 24 hours) while also depending on the capacity of the canister for CO2 absorption, and hence depending on the amount of CO2 concentration present or captured by the canister after one day. Moreover, the more CO2 in the breathing system, the more needs to and will be captured and absorbed by the CO2 removal device.

It is further noted that, in case only one single CO2 removal device is used in the breathing system, the operational settings may be such that discharging goes faster than charging. In other words, it would probably be desirably then, that the charging on-status of the CO2 removal device is much longer than the off-status when discharging can take place, and hence not much time is lost or spent during the discharging operation.

1-14. (canceled)

15. A device for CO2 removal from an expiration or breathing gas stream, suitable for use in a breathing system, part of an anaesthesia arrangement, the device comprising:
a holder adapted for fitting in the breathing system; and
a container for CO2 removal from a gas stream, the container being adapted for fitting in the holder,
wherein:
the CO2 removal by the container is based on an electro-charging and discharging method,
the container comprises a plurality of plates arranged in that the expiration or breathing gas stream passes between the plates;
each of the plates comprises at least two electrodes arranged for charging and/or discharging the plates; and
the at least two electrodes comprise an active electrode and a counter electrode separated from the active electrode by a separator; and
the device further comprises means for providing electricity to the active electrode and/or the counter electrode.

16. The device of claim 15, wherein the plurality of plates are adapted for absorbing CO2 when the electrodes are being charged and for releasing CO2 when the electrodes are being discharged.

17. The device of claim 15, further comprising electronic means to control charging or discharging of the electrodes.

18. The device of claim 15, adapted for having a pressure less than 2 mbar at a nominal flow rate from 20 L/min to 65 L/min, and comprising at least two plates, wherein a distance d between the plates or the electrodes thereof is selected therefor.

19. The device of claim 15, wherein a surface of the electrodes is selected to react with CO2 in the gas stream.

20. The device of claim 19, wherein the surface of the electrodes is covered with a polymer, the polymer comprising a material for attracting CO2.

21. The device of claim 20, wherein the material for attracting CO2 is (anthra)quinone.

22. The device of claim 21, adapted to be operable for concentrations of CO2 as low as about 400 ppm by selecting an amount of (anthra)quinone and a total surface applicable in the container.

23. The device of claim 19, wherein the surface of the electrodes is covered with a polymer, the polymer comprising a material for enhancing conductivity of the electrodes.

24. The device of claim 23, wherein the material for enhancing conductivity of the electrodes comprises carbon nanotubes.

25. The device of claim 19, wherein the surface of the electrodes is covered with a polymer, the polymer comprising (anthra)quinone for attracting CO2 and carbon nanotubes for enhancing conductivity of the electrodes.

26. The device of claim 15, adapted for being operable for at least one day, by adjusting a number of the plates in the container for CO2 removal.

27. The device of claim 15, adapted to be operable for at least 2000 charging-discharging cycles with less than 30% efficiency loss of charging-discharging operation.

28. A breathing system comprising the device for CO2 removal from a gas stream according to claim 15.

29. An anaesthesia arrangement comprising the breathing system according to claim 28.

30. An electro-charging and discharging method for operating the device according to claim 15 for CO2 removal from an exhaustion or breathing gas stream in a breathing system as part of an anaesthesia arrangement, the method comprising:
when the device is installed in a gas flow circulation of the breathing system from which CO2 is to be removed, providing electricity of a first polarity to the container to charge the electrodes; and
when the device is removed from the gas flow circulation, providing electricity of a second polarity opposite the first polarity to the container to discharge the electrodes.

31. A discharging equipment, adapted to receive the device according to claim 15 and to provide electricity of a suitable polarity for discharging the device.
